(19) United States
(12) Patent Application Publication
(10) Pub. No.: US 2007/0117750 A1
(43) Pub. Date: May 24, 2007

(54) METHOD FOR ENHANCED OCULAR DRUG PENETRATION

(76) Inventor: Muhammad ABDULRAZIK, East-Jerusalem (IL)

Correspondence Address: Edward Langer;C/O Shiboleth, Empire State Building, 60th Floor, 350 Fifth Avenue, New York, NY, 10118, US

(21) Appl. No.: 11/538,836

(22) Filed: Oct. 5, 2006

(30) Foreign Application Priority Data
Oct. 6, 2005 (IL) 171271

(51) Int. Cl. A61K 38/18; A61K 38/13; A61K 31/7048; A61K 31/704; A61K 31/573; A61K 31/57; A61K 31/554; A61K 31/553

(52) U.S. Cl. 514/12; 514/171; 514/11; 514/291; 514/29; 514/27; 514/454; 514/211.07; 514/649; 514/449; 514/410; 514/34; 514/254.07; 514/263.31

(57) ABSTRACT

Provided is a method for enhanced intraocular drug penetration that comprises the co-administration of ocular therapeutics with agents that increases the permeability of ocular and periocular vessels and ocular epithelial barriers. Due to its unique and novel concept and additive nature, the method of the present invention can be used in combination with previous methods for enhancement of ocular drug penetration.

### FIELD OF THE INVENTION

The invention relates generally to methods for enhanced ocular drug penetration and, more specifically, the invention relates to the co-administration of ocular therapeutics with agents that increases the permeability of ocular and periocular vessels and ocular epithelial barriers by unique strategy and thus with additive ocular penetration enhancement effect that allows the utilization of this invention in combination with known methods for ocular drug penetration enhancement.

### BACKGROUND OF THE INVENTION

The following is a brief overview of eye anatomy that, together with the schematic description in FIGS. 1 and 2, is needed to understand the bellow discussion on obstacles to intraocular drug penetration.

The ocular surface consists of the superficial layers of the cornea and conjunctiva. It is well accepted that one of the main tasks of the ocular surface is to create a defense barrier against penetration from undesired molecules. In humans the corneal surface is only 5% of the total ocular surface while the remaining 95% is occupied by the conjunctiva.

The cornea is made up of 5 layers (epithelium, Bowman's layer, stroma, Descemet's membrane, and endothelium), but only the outermost 2-3 layers of the corneal squamous epithelial cells form a barrier for intercellular drug penetration by means of intercellular zonulae ocludents. The superficial epithelial layer of the conjunctiva is continuous with the corneal epithelium and stretches deeply to form the conjunctival sacs and back to join the epidermis of the lids. The stratified epithelium varies in thickness from 2 to 4 layers at the junction with eyelid margin skin, to 6 to 8 layers at the junction with corneal epithelium. The substantia propria of the conjunctiva consists of superficial lymphoid layer and a deeper fibrous layer, a thick collagenous and elastic tissue that is rich with blood vessels.

The skeleton of the eye globe consists of the rigid corneal-scleral collageneous shell. The sclera covers the posterior 80% of the eye globe except for a small posterior opening occupied by the optic nerve head, while the rest of the globe is covered anteriorly by the cornea. The scleral stroma is composed of bundles of collagen, fibroblasts, and a moderate amount of ground substance. This tissue is essentially a vascular but is lined superficially by the vascular episclera. A large number of channels penetrate the sclera to allow the passage of vessels and nerves to the choroid side. In humans, the scleral thickness is in the range of 0.3-1.0 mm with the posterior pole being the thickest.

The iris is made up of connective tissue and blood vessels, with variable contents of melanocytes and pigment cells that are responsible for its color. The posterior surface of the iris is lined by a monolayer of iridial pigment epithelium (IPE) that is continuous peripherally with the nonpigmented epithelium of the ciliary body. The iris diaphragm subdivides the anterior segment of the eye into the anterior and posterior chambers. The pupil is a round opening in the center of the iris diaphragm that allows the passage of light towards the retina and aqueous humor from the posterior to the anterior chamber and its diameter is changed by the action of the iris sphincter and dilator muscles.

The ciliary body is a triangular structure at the periphery of the border between the anterior and posterior segments of the eye. Its uveal portion consists of comparatively large fenestrated capillaries, collagen fibrils, and fibroblasts. The ciliary body is lined by a double layer of epithelial cells, the nonpigmented and the pigmented epithelium with their apices facing each other. The basal side of the inner nonpigmented epithelium is embedded in the aqueous humor of the posterior chamber. The apical borders of the nonpigmented epithelium are sealed with zonulae-occludentes that maintain the blood-aqueous barrier.

The choroid is a highly vascularized tissue, with one of the highest blood flow rates among body tissues. Beside vessels and intercellular space collagen fibers, choroidal stroma consists of abundant melanocytes as well as variable contents of macrophages, lymphocytes, mast cells, and plasma cells. In the human eye this layer thickness averages 0.25 mm and consists of an innermost layer of fenestrated choriocapillaris, middle medium and outer large vessels (both non-fenestrated). Bruch's membrane separates the choroid from the retinal pigment epithelium and consists of a series of connective tissue sheets, including the basement membranes of the retinal pigment epithelium and the choriocapillaries.

The retina consists of ten layers: the retinal pigment epithelium, photoreceptor outer segments, external limiting membrane, outer nuclear layer, outer plexiform layer, inner nuclear layer, inner plexiform layer, ganglion cell layer, nerve fiber layer, and internal limiting membrane.

The retinal pigment epithelium (RPE) consists of a monolayer of hexagonal cells of neuroectoderm origin that starts from the optic nerve head posteriorly and extends until it merges with the pigmented epithelium of the ciliary body anteriorly. Adjacent RPE cells are firmly attached by zonulae occludentes that play an important role in maintaining the outer blood-ocular (retinal) barrier (oBOB). The apical side of the RPE has numerous villous processes and faces the subretinal space. The basal side faces the choroid and is attached to a thin basement membrane that forms the inner layer of Bruch's membrane.

The eye globe is a hollow structure filled anteriorly by the aqueous humor and posteriorly by the vitreous cavity. The aqueous humor is continuously formed by the ciliary body. It is secreted into the posterior chamber from which it passes through the pupil into the anterior chamber and is drained at the anterior chamber periphery. Due to the blood-aqueous barrier formed by zonulae ocludents of the nonpigmentary ciliary epithelium, macromolecules such as proteins can pass to the aqueous in very small quantities, regardless of their plasma concentrations.

The vitreous occupies 80% of the volume of the human eye, and its volume is approximately 4.0 ml. The vitreous consists of hyaluronic acid, collagen fibrils and hyalocytes. Even though the water content of the vitreous is around 99%, this tissue has a gel-like structure and its viscosity is approximately twice that of water. The gel-like vitreous can be liquefied in a disease and aging processes. Drugs move in the vitreous by diffusion through the gel when it is formed and by convection when it is liquefied.

Routes of Drug Permeation to Intraocular Tissues

The ciliary body (currently the main target tissue in anti-Glaucoma therapy) and the retina are the ultimate target tissues for pharmacological interventions in modern ophthalmology. Both are intraocular tissues that necessitate efficient drug permeation through different barriers before being reached following local or systemic drug administration.

For decades, researchers adhered to a two-route model to describe the penetration of topically applied drugs to the intraocular tissues. In this model the corneal route comprises the penetration through the cornea to the aqueous humor, while the conjunctival route comprises the penetration through the conjunctiva to the sclera.

Traditionally the importance of the corneal route was overestimated mainly because aqueous humor concentration of a drug was considered to represent the intraocular availability of that drug, and because of the misconception that drug found in the aqueous humor originated solely from drug permeation through the cornea. In fact many researchers used the term “non-productive absorption” to describe most of the conjunctival absorption.

The possibility that drug in the aqueous humor could originate not only from transcomeal penetration, and that drug absorbed by the noncorneal route could reach intraocular tissues by means other than the aqueous humor of the anterior chamber, was raised two decades ago. Maurice [Maurice D M. in Pharmacology of the eye, Sears M L ed., Springer & Verlag, New York, 1984:19-116] suggested that drugs, once absorbed into the conjunctiva, diffuse laterally into the scleral border of the cornea and then directly enter the aqueous humor. Additional research reported that the noncorneal absorption route may be important for drugs that are poorly absorbed across the cornea due to their physical-chemical properties. Aqueous humor timolol was found to originate from transcorneal penetration while most of the drug found in the iris/ciliary body was related to conjunctival/scleral penetration. Chein et al. [Chien D S et al. Curr Eye Res. 1990; 9:10511] studied the ocular penetration pathways of three alpha 2-adrenergic agents. In contrast to the more lipophilic drugs where the corneal route was the predominant pathway for delivering drugs to the iris, the less lipophilic brimonidine underwent conjunctival/scleral penetration for its delivery into the ciliary body.

Obstacles for the Permeation of Locally Applied Therapeutics

The superficial 2-3 cell layers of the corneal and conjunctival epithelium are the main barrier for permeation of topically applied compounds. In this rate-limiting cell layer, the transcellular permeation is dictated by the biophysical character of the cell membrane while the paracellular permeation is limited by the paracellular pore size and density.

Cell membrane has a lipophilic character that functions as a selective barrier denying free diffusion from hydrophilic compounds. The encircling of cell contents by a cell membrane is an important evolutional milestone aimed at protecting cell contents from external environment and allowing the creation of an autonomic biological microsystem to carry out undisturbed precise biological reactions. The evolutional rationale of protecting biological microsystem by a lipophilic membrane might be that the main threat to the normal function of such systems comes from bioactive molecules like enzymes, peptides and oligonucleotides rather than from lipophilic compounds. However, evidence has emerged in recent decades showing the existence of defense mechanisms against lipophilic molecules as well. Several studies have reported the expression of active drug extrusion pumps (multidrug-resistance protein 1, p-glycoprotein) in the surface epithelium of the cornea and conjunctiva, which are responsible for the extrusion of some lipophilic drugs from the surface epithelium back to the tear film.

Reports have been made that paracellular pores are fewer (6%) and their size is smaller (50%) in the cornea as compared to the conjunctiva. The total paracellular space in the conjunctiva was estimated to be 230 times greater than that in the cornea. Indeed, the conjunctiva was reported to be 15 to 25 times more permeable to polyethylene glycols (PEGs) than the cornea. In the cornea the reported paracellular pore density was 4.3×10⁶/cm² with an estimated pore diameter of 2.0 nm, corresponding to a permeation facility for molecules with a Stokes' radius of 1.0 nm. The paracellular conjunctival epithelial pores measured approximately 3.0 nm in the bulbar conjunctiva and 4.9 nm in the palpebral conjunctiva, allowing the potential permeation of molecules with 1.5 nm and 2.5 nm Stokes' radius respectively. Therefore, theoretically peptides and oligonucleotides with molecular weights of 5 kDa can permeate through the bulbar and palpebral conjunctiva while those with molecular weights of 10 kDa can permeate only through the palpebral conjunctiva. However, experiments have shown that there was no significant difference in the permeability of palpebral and bulbar conjunctiva to PEGs. Ex-vivo ocular surface permeability for topically applied hydrophilic macromolecules (4 kDa and 10 kDa dextrans) was shown experimentally to be markedly low, although the permeability for the conjunctiva was much higher compared to the cornea.

In the corneal route, after permeation through the corneal epithelium, molecules should then permeate through the hydrophilic corneal stroma and the corneal endothelium before reaching the aqueous humor. Thus, while the corneal epithelium favors the penetration of lipophilic compounds, the hydrophilic stroma favors the permeation of hydrophilic compounds. The corneal endothelium monolayer is not sealed by paracellular zonulae occludentes. Consequently this layer has only a minimal effect on the overall permeability of the cornea. Overall, the cornea as a whole is tailored to allow the permeation of moderately lipophilic compounds (close to log P=2). On the contrary, the reported optimal lipophilicity for compounds penetrating through an isolated epithelial layer of the cornea was around log P of 4 [Civiale C et al. J Ocul Pharmacol Ther. 2004; 20:75].

In any case, drug availability in aqueous humor does not ensure availability at more remote target tissues like the ciliary body and the retina. In the human eye, the volume of the anterior chamber is 200-250 μL and the rate of aqueous flow rate is at the level of 2.5 μL per minute in average. Drugs available in the aqueous humor are subject to an elimination process that is proportional to the aqueous turnover rate. Furthermore, the backward (toward the retina) permeation facility from the anterior to the posterior chamber through the narrow and apparently impermeable pupil-lens canal and against the current of the aqueous humor, or through the lens itself, is debatable [Maurice D M. Surv Ophthalmol. 2002; 47(Suppl 1):S41].

In the conjunctival route, after permeation through the conjunctival epithelium, molecules should then permeate through the sclera before proceeding towards the ciliary body and the retina. The permeability of the sclera is comparable to that of corneal stroma. Published reports have shown that bovine, human, and rabbit sclera are permeable even to high molecular weight compounds [(Maurice D M and Polgar J. Exp Eye Res. 1977; 25:577), (Olsen T W et al. Invest Ophthalmol Vis Sci. 1995; 36:1893)].

Ambati et al. [Ambati J et al. Invest Ophthalmol Vis Sci. 2000; 41:1181] measured the permeability of rabbit sclera to a series of fluorescein-labeled hydrophilic compounds and found that scleral permeability decreased with increasing molecular weight and molecular radius but was quite permeable to large molecules, such as IgG (145 kDa). Thus when targeting the retina by the topical or subconjunctival routes the sclera is not a rate-limiting barrier and the main barriers are beyond this tissue, most probably at the vascular bed of the choroid and the encircling barrier line of the retinal pigment epithelium monolayer.

Obstacles Facing Locally Diffusing Compounds Approaching the Vitreous-Retina Compartment

The retina is an immune privileged tissue situated in a highly protected compartment with multiple defense lines. Like brain neurons, the retinal neurons need strictly controlled homeostasis for their proper function. Selective barriers separate, therefore, the retina from the adjacent tissues and the blood circulation. The vitreous-retina compartment occupies most of the intraocular volume and is encircled almost 360 degrees by a series of epithelium monolayers with paracellular zonulae occludentes. This encircling barrier includes the retinal pigment epithelium (RPE) posteriorly, the iris posterior pigmented epithelium (IPE) anteriorly, and the non-pigmented epithelium of the ciliary body (CNPE) in between. The pupil and the optic nerve head are the only gaps in this defensive encircling. However, drug diffusion through the pupil is very low due to the narrow and apparently impermeable iris-lens canal, the counter-flow of the aqueous humor from the posterior to anterior chamber, the relatively impermeable lens, and the dense zonular diaphragm that connects the lens with the ciliary processes of the ciliary body. Furthermore, the bulk of the vitreous is a dilutional and to some extent metabolic obstacle that faces drugs on their way from the anterior vitreous border to the posterior retina.

Therefore, the encircling epithelial monolayer with paracellular zonulae occludentes provides a selective barrier separating the whole vitreoretinal compartment from the rest of the eye. Therefore, only molecules that can permeate through this encircling barrier line can reach the retina.

Reportedly [Pitkanen L et al. Invest Ophthalmol Vis Sci. 2005; 46:641], the isolated bovine RPE-choroid was up to 20 times more permeable to lipophilic than hydrophilic beta-blockers. Furthermore, the in-vitro permeability of bovine RPE-choroid to hydrophilic compounds and macromolecules was 10 to 100 times less compared to sclera, while the permeability for lipophilic molecules was in the same range for both tissues. The isolated bovine RPE-choroid also exhibited differential permeation by molecular weight and Stokes radius. The permeation rate of 4, 10 and 20 kDa fluorescein labeled (FITC) dextrans was moderate compared to a good permeation rate for the 376 Dalton carboxyflourecein and a poor penetration rate for 40 and 80 kDa FITC-dextrans. The permeability to carboxyfluorescein was 35 times more than to 80 kDa FITC-dextran [Pitkanen L et al. Invest Ophthalmol Vis Sci. 2005; 46:641]. In a study on the permeability of the human ciliary epithelium to a horseradish peroxidase (PO), Tonjum and Pedersen [Tonjum A M and Pedersen O O. Acta Ophihalmol (Copenh) 1977; 55:781] reported that ciliary and iridial epithelium contained a system of paracellular zonulae occludentes. PO was applied on the stromal side of ciliary body and iris specimens obtained from freshly enucleated eyes. The 40 kDa PO was blocked apically in the lateral intercellular spaces of the CNPE while in the iris the progression of PO was blocked apically in the lateral intercellular spaces of the IPE. Freddo [Freddo T F. Invest Ophthalmol Vis Sci. 1984; 25:1094] studied the intercellular junctions in the posterior IPE cells of the rhesus monkey by electron microscopy, freeze-fracture and horseradish peroxidase. Intravenously injected horseradish peroxidase, which diffused from the ciliary body stroma, was prevented from reaching the posterior chamber by the presence of the zonulae occludentes between adjacent posterior IPE cells. The author concluded that these apico-lateral zonulae occludentes are analogous in both location and degree of permeability to the zonulae occludentes present in the CNPE. In another study the functional barrier for macromolecules in IPE and RPE cells was reported to be similar in an in vitro setting [Rezai K A et al. Graefes Arch Clin Exp Ophthalmol. 1997; 235:48].

Obstacles Facing Blood-Derived Compounds Approaching the Vitreous-Retina Compartment

Blood-derived compounds can reach the retina trough the uveal (choroidal) circulation or through the retinal blood vessels. As expected from the above discussion, blood-derived compounds approaching the retina from the fenestrated choroidal vasculature face the obstacle of the RPE monolayer, which forms the outer blood-ocular (retinal) barrier (oBOB), while blood-derived compounds reaching the retina through the retinal vessels face the obstacle of the inner blood-ocular barrier (iBOB) formed by the CNS-like retinal vasculature. The retinal blood vessels are analogous to the cerebral blood vessels, expressing blood-brain-barrier like properties. Stewart and Tuor [Stewart P A. and Tuor U I. J Comp Neurol. 1994; 22:566] have reported the similarity of retinal and brain vasculature permeability in the rat. Compared to the brain vessels the more porous endothelium of the retinal vessels is compensated by a four-fold denser pericytes layer. This inner blood-ocular barrier (iBOB) consists of a single layer of nonfenestrated endothelial cells with tight junctions. The endothelial layer is covered by a basal lamina with an interrupted layer of pericytes embedded in the surrounding basement membrane matrix. In humans, retinal vessels are impermeable to tracer substances such as fluorescein and horseradish peroxidase (40 kDa) [Park S S, Sigelman J, and Gragoudas E S. in Duane's Ophthalmology on CD-ROM, Tasman W and Jaeger E A eds., LW&W. 2002; vol. 1:chap 1].

In the prior art, the RPE together with the scleral/episcleral and choroidal vasculature were recognized as a significant obstacle that critically limits the permeation of drugs to the retina when these drugs were administered extraocularly, either topically or subconjunctively. Nevertheless, the role of the ocular vascular beds in delivering or diverting topically applied drugs to or away from the target tissue remained debatable. Chiang and Schoenwald [Chiang C H and Schoenwald R D. J Pharmacokinet Biopharm. 1986; 14:175] have shown that following topical instillation of clonidine, peak concentrations were reached in the iris ciliary/body at 2 minutes or at the first time interval that can be measured, while the peak concentration time for the aqueous humor was 15 minutes. These results imply that drug penetration to the ciliary body and iris is mediated via a fast delivery route that probably is a vascular one. Schoenwald et al. [Schoenwald R D et al. J Ocul Pharmacol Ther. 1997; 13:41] studied the uptake and distribution of drugs by vessels in the rabbit eye following topical instillation. Following entry of drug into the conjunctival/scleral tissue a significant portion of the drug entered scleral vessels, as evidenced by the confocal laser scanning microscopy, and reached the ciliary body. On the contrary, it have been reported that topically applied drugs, with assumable penetration into the conjunctival vasculature, were diverted from ocular tissues to the systemic circulation [Vuori M L et al. Acta Ophthalmol. (Copenh) 1993; 71:682]. Flurbiprofen, a nonsteroidal antiinflammatory agent which is not ocularly metabolized, was used as a probe to show the proportion of topically applied drug lost to the systemic circulation following uptake by ocular and local vascular beds. The systemic bioavailability of flurbiprofen following the administration of topical ocular dose of 225 μg was 74% [Tang-Liu D D, Liu S S, and Weinkam R J. J Pharmacokinet Biopharm. 1984; 12:611].

In the past, attempts were made to overcome the possible diverting obstacle of local vascular beds by co-administration of vasoconstrictors with ocular therapeutics. However, the beneficial effect of the co-administered vasoconstrictors was related mainly to the vasoconstriction of the vascular bed of the nasal mucosa (Jarvinen K, Urtti A. J Ocul Pharmacol. 1992; 8(2):91-8) and not the deep episclera/scleral or choroidal vessels. Indeed, a similar beneficial effect to that of vasoconstrictors co-administration was simply achieved by nasolacrimal occlusion (Sharir M, Zimmerman T J. J Assoc Acad Minor Phys. 1994; 5(2):62-7). Furthermore, in a MRI imaged pharmacokinetic study, comparing legend penetration from the subconjunctival space to the intraocular space between in-vivo (with intact blood circulation) and postmortem conditions (with no blood circulation), vasculature clearance by episcleral/scleral vessels (and perhaps choroidal vessels as well) was identified as a main barrier for intraocular permeation (Li S K, Molokhia S A, Jeong E K. Pharm Res. 2004; 21(12):2175-84).

In order to avoid the hazardous intravitreal pharmacological interventions, the prior art have witnessed several attempts to enhance the intraocular drug penetration by non-invasive or minimally invasive methods. These approaches included efflux inhibition, influx transport enhancement, chemical penetration enhancement, physical penetration enhancement, biological penetration enhancement, drug encapsulation technology, drug reservoir technology and drug convection or pumping technology. However, the existing approaches for delivering drugs to intraocular tissues suffer from significant and well recognized limitations. Therefore, the need for effective and minimally invasive method for intraocular drug penetration remained unmet.

### SUMMARY OF THE INVENTION

In the method of the present invention, the co-administration of placental growth factor (PlGF) with model-drug, either simultaneously or at different administration times, was evidenced to cause a temporary leak in the orbital, episcleral/scleral and choroidal vessels and the consequent release of the model-drug cleared by these vessels at sites with intimate proximity to the target tissues. Furthermore, the same PlGF intervention was evidenced to lead to increased permeability of the RPE barrier and consequently to enhanced model-drug penetration towards the retina.

Therefore, in view of the limitations of existing ocular drug delivery systems, it is an object of the present invention to provide a method for enhanced ocular drug penetration that allows for effective concentrations of ocular therapeutics in intraocular tissues.

It is an object of the present invention to provide a method and composition that will improve and enhance the process of drug penetration to ocular and periocular tissues in a unique and novel way that overcomes the obstacle of ocular surface barriers and blood-ocular barriers.

This object was accomplished by the co-administration of ocular therapeutics with a factor that can increase the permeability of ocular surface and blood-ocular barriers.

For the purpose of this invention, the factor that can increase the permeability of ocular surface and blood-ocular barriers is preferably a stimulator of receptor-1 of the vascular endothelial growth factor (VEGFR-1 or Flt-1).

Example of a stimulator of VEGFR-1, which may be preferentially co-administered with ocular therapeutics, is an effective amount of placental growth factor (PlGF).

For the purpose of this invention, PlGF is defined as any isomer of this growth factor like the following examples: PlGF-1 (PlGF₁₃₁), PlGF-2 (PlGF₁₅₂) and PlGF-3 (PlGF₂₀₃).

The object of the present invention can be accomplished by either simultaneous or non-simultaneous co-administration of the ocular therapeutic agent and the factor that can increase the permeability of ocular surface and blood-ocular barriers, as dictated by the time table that leads to an optimal effectiveness.

For the purpose of this invention, the co-administration of the factor that can increase the permeability of ocular surface and blood-ocular barriers can be done in several ways that can be selected without limitation from the following: topical ocular, subconjunctival, periocular, intracameral and intravitreal routes of administration. Similarly, the administration of the ocular therapeutic agents can be done in several ways that can be selected without limitation from the following: topical ocular, subconjunctival, periocular and any non-ocular route of administration.

It is a further object of the present invention to utilize the unique and highly selective concept of innovation, that promises additive penetration enhancement effect with most previously described methods for enhanced ocular drug penetration, in order to suggest combinations of the method of the present invention with known methods of penetration enhancement. These known methods of penetration enhancement is selected without limitation from the following methods: efflux inhibition, influx transport enhancement, chemical penetration enhancement, physical penetration enhancement, biological penetration enhancement, drug encapsulation technology, drug reservoir technology and drug convection or pumping technology

Other objects, features and advantages will be apparent from the following detailed description of the invention.

### BRIEF DESCRIPTION OF THE DRAWINGS

For a better understanding of the invention with regard to the embodiments thereof, reference is made to the accompanying drawings, wherein:

FIG. 1 depicts a schematic drawing of the eye, showing three different views: the lower part of the Figure is a diagram of the entire eye; the two upper diagrams illustrate enlarged portions of the eye.

FIG. 2 shows a schematic drawing of the main barriers of the eye, showing three different views: the lower part of the Figure is a general diagram of eye posterior segment. The two upper diagrams illustrate enlarged portions of the eye posterior segment.

### DETAILED DESCRIPTION OF THE INVENTION

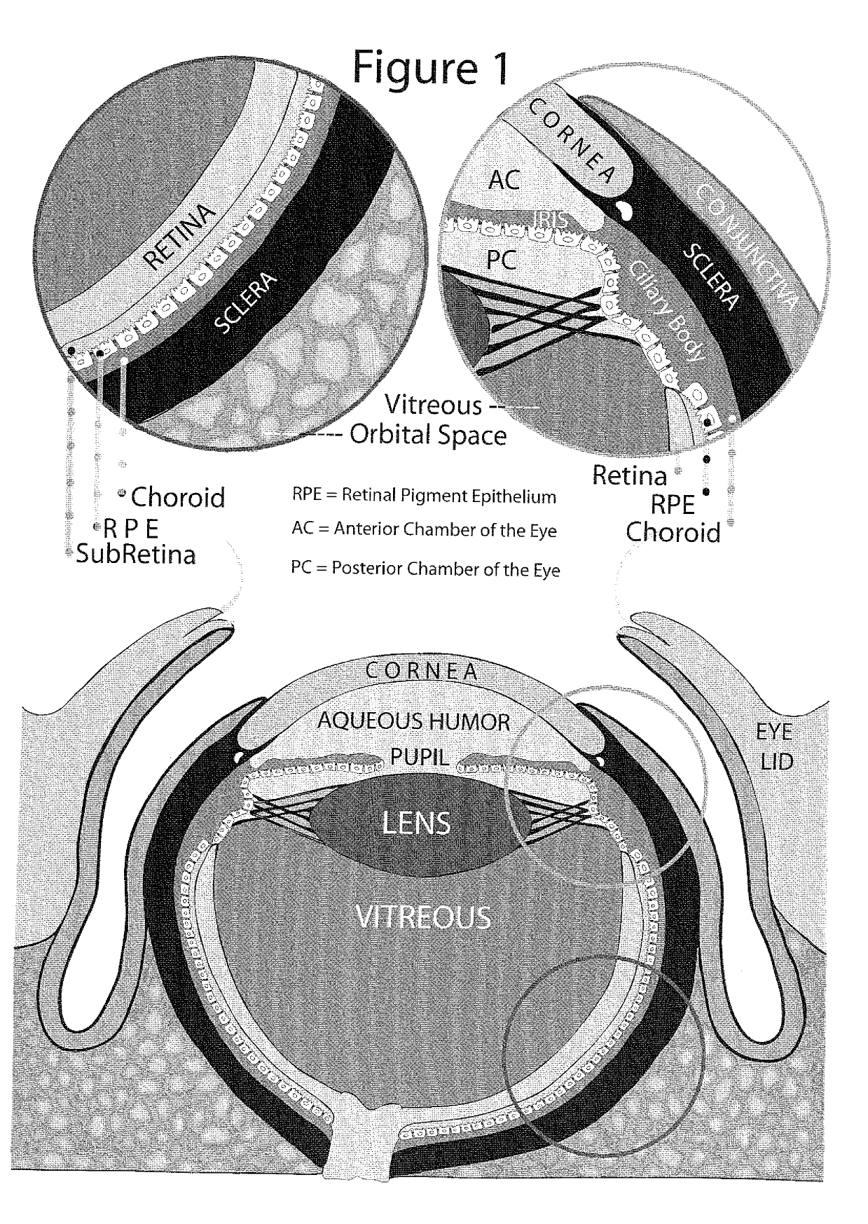

FIG. 1 shows a schematic illustration of the eye and surrounding tissues. It is intended to show the ocular barriers that deny intraocular penetration from locally administered therapeutics. The illustration shows that the retina is the most secured organ in the eye with multiple barriers that ensures a strict isolation of this tissue, a tough challenge facing all drug delivery strategies suggested so far to target the retina. Surrounding the retina is the choroid, the RPE (retinal pigment epithelium) and the subretina layers, as well as the sclera, as seen in the diagram located in the upper left of FIG. 1. Other eye parts shown in the lower diagram are the cornea, the aqueous humor, the pupil, the lens, and the vitreous space. In the upper right diagram of FIG. 1, it is possible to see the anterior and posterior chambers of the eye, the conjunctiva at the outer edge of the eye, the cornea, the sclera surrounding the cornea, the iris, and the ciliary body around the iris.

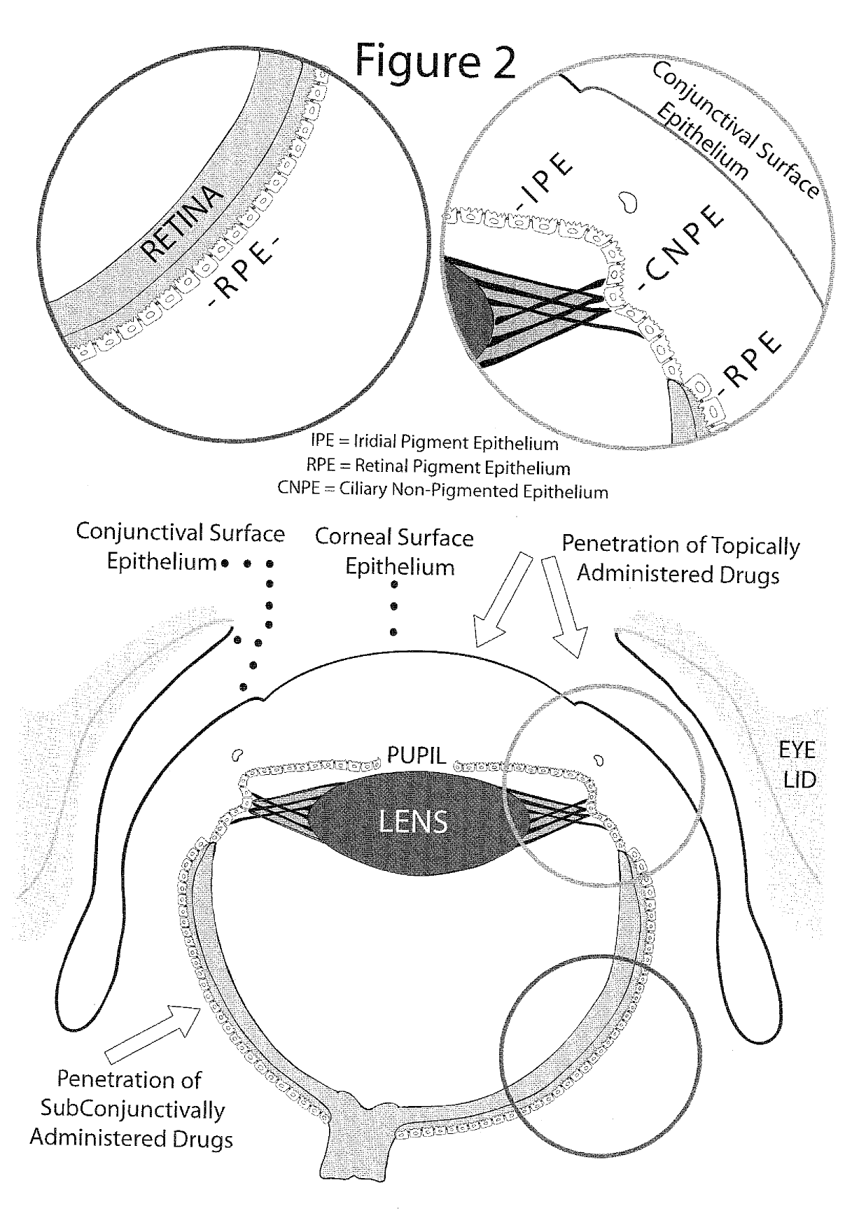

FIG. 2 shows a reduced schematic illustration of the eye and surrounding tissues that shows the significant ocular barriers that protect the retina. The illustration shows that significant barriers face not only drugs administered topically but subconjunctively administered drugs as well. The illustration shows the critical role of the RPE defense line in denying penetration from drugs administered subconjunctively. Drugs that are administered subconjunctivally have to pass through the RPE. Other layers protecting the eye are the IPE (iridial pigment epithelium), and the CNPE (ciliary non-pigmented epithelium), shown enlarged in the diagram in the upper-right of FIG. 2. The conjunctival surface epithelium and the corneal surface epithelium surround the outer eye.

The invention relates to an improved method for enhanced ocular drug penetration that allows for effective concentrations of ocular therapeutics in intraocular tissues.

The invention is based on a novel concept that integrates experimental findings with grows hormones, that were found to exhibit unexpected effects on vascular and ocular barriers, and thorough understanding of vascular and barriers anatomy and physiology of the eye and its surrounding tissues.

When the drainage to the nose is avoided by nasolacrimal occlusion and spillage to the skin of eyelids and cheeks is prevented by avoiding the administration of excessive volumes of drug solution, the anatomy of ocular vessels is such that most of the vascular drug clearance is done through the ophthalmic veins, that runs backwards through the orbit and towards the cavernous sinus with intimate proximity to the eyeball walls, rather than outward through the facial veins.

Indeed, when the method of the present invention was used it was evidenced to cause a temporary leak in the orbital, episcleral/scleral and choroidal vessels, and the consequent release of the model-drug cleared by these vessels, at sites with intimate proximity to the target tissues. Together with the evidenced increased permeability of the epithelium barriers, these consequences of the PlGF intervention have led to enhanced model-drug penetration towards the retina and other intraocular target tissues.

Placental growth factor (PlGF) was discovered in human placenta in 1991, about 2 years after the identification of vascular endothelial growth factor (VEGF), and was so named because it is abundant in placental tissue (Maglione D et al. Proc Natl Acad Sci USA 1991; 88:9271-6).

This dimeric glycoprotein, with 3 identified isomers which differ in size and binding properties (PlGF-1 (PlGF131), PlGF-2 (PlGF152) and PlGF-3 (PlGF203)), was later identified as a member of the VEGF family as the molecule shares about half of a homologous domain with the platelet derived growth factor-like region of VEGF (Cao Y, et al. Biochem Biophys Res Commun 1997; 235(3):493-8).

PlGF homodimers exclusively bind with high affinity to the tyrosine kinase VEGF receptor-1 (Flt-1), but have little effect on angiogenesis in vitro. Indeed, PlGF had a negligible role in physiological angiogenesis and vascular development but do play a role in pathological angiogenesis. PlGF and VEGFR-1 are minimally expressed in adult quiescent vasculature, but are markedly upregulated during pathological conditions (Carmeliet P et al. Nat Med. 2001; 7(5):575-83).

Shih et al have suggested the activation of VEGFR-1 by PlGF-1 is for preventing oxygen-induced retinal ischemia. This study has demonstrated that PlGF-1 intervention didn't carry the risk of provoking retinal neovascularization (Shih S C et al. J Clin Invest. 2003; 112(1):50-7).

In a study of on the hydraulic conductivity of frog mesenteric capillaries, Hillman et al. have reported that the stimulation of VEGF receptor-2 by VEGF homodimers have caused a chronic increase in microvascular permeability. On the contrary, the stimulation of VEGF receptor-1 by PlGF-1 homodimers didn't cause a long lasting effect on microvascular permeability (Hillman N J et al. J Vasc Res. 2001; 38(2):176-86).

Xu et al. have reported that Tumors overexpressing PlGF in the mouse have exhibited higher levels of PlGF homodimers and PlGF/VEGF heterodimers but decreased the levels of VEGF homodimers. Consequently, there was a decrease in tumor angiogenesis, growth and metastasis, all being effects related to VEGF homodimers. The authors have postulated that this effect of PlGF overexpression was achieved by the depletion of VEGF homodimers due to the formation of PlGF/VEGF heterodimers (Xu L et al. Cancer Res. 2006; 66(8):3971-7).

Ghassemifar and coworkers have studied the effect of VEGF on tight junctions formation between retinal pigmented epithelial (RPE) cells by examining two essential proteins, ZO-1α+ and ZO-1α−. They have reported that in the presence of 5 ng/ml VEGF, cultured RPE cells (ARPE19 and RPE51) significantly up-regulate ZO-1α+ and ZO-1α− transcripts and proteins resulting in a significant increase in their TER. (Ghassemifar R, Lai C M, Rakoczy P E. Cell Tissue Res. 2006; 323(1):117-25).

The work of Xu, when taken together with the work of Ghassemifar, teach that acute PlGF intervention will temporarily increase the permeability of the RPE cells without chronic sequel, presumably through downregulated intercellular tight junction expression. However, the applicant has evidenced the effects of PlGF on the permeability of RPE cells and ocular vessels well before the report of Xu, while the work of Ghassemifar didn't include any disclosure about PlGF.

In one embodiment of the method of the present invention, a PlGF dose is prepared prior to its administration from mother solution that is kept in the conditions recommended by the manufacturer. The ocular therapeutic solution is prepared according to the nature of the particular drug with the addition of the necessary pharmaceutical exepients. The administration of both solutions could be simultaneous or at different times, subject to penetration effectiveness studies of a particular drug. The administration of the PlGF solution and the solution that contains the desired ocular therapeutic could be administered topically if the target tissue is in the anterior segment of the eye. Alternatively, administration to the anterior subconjunctival space will be preferable if targeting of the ciliary body, the anterior retina or the corneal endothelium is desired. In another alternative, administration to the posterior subconjunctival space is preferred for targeting the more posterior portions of the retina.

In another embodiment of the present invention, the administration sites for the PlGF and the desired ocular therapeutic agent could be different. In one possibility, the PlGF could be administered into the vitreous while the therapeutic agent is provided subconjunctively. In another possibility, the PlGF could be administered into the vitreous while the therapeutic agent is provided through non-ocular site. In addition, the PlGF could be administered subconjunctively while the therapeutic agent is administered topically or vice versa.

The implementation of the method of the present invention is not limited to a certain class of ocular therapeutics. The issue of drug penetration to the retina is emphasized in the description of the present invention, simply because the targeting of this ocular tissue is currently one of the most challenging drug delivery problems facing experts in the field. Obviously, the method of the present inventions is applicable to any target tissue of the eye.

The unique and novel concept of the present invention promises additive enhancement effect when the method of the present invention will be used in combination with known methods for the enhancement of ocular drug penetration.

Therefore, the method of the present invention may be used additively with an adjuvant method for enhanced ocular drug penetration. Adjuvant methods that could be used additively with the method of the present invention are selected without limitation from the following list: efflux inhibition, influx transport enhancement, chemical penetration enhancement, physical penetration enhancement biological penetration enhancement, drug encapsulation technology, drug reservoir technology and drug convection or pumping technology.

According to preferred embodiments of the present invention, efflux inhibition is by the utilization of a p-glucoprotein inhibitor. The glucoprotein inhibitor is preferably an agent selected from, but not limited to, verapamil, bepridil, dilatiazem, felodipine, nifedipine, nisoldipine, nitrendipine, tiapamil, vincristine, actinomycin D, coichicines, daunorubicin, etoposide, mitomycin C, mithramycin, podophyllotoxin, puromycin, taxol, topotecan, triamterene, vinblastine, cyclosporine A, cyclosporine H, tacrolimus, sirolimus, aldosterone, clomiphene, cortisol, deoxycorticosterone, dexamethasone, prednisone, progesterone analogs, tamoxifen, hydrocortisone, testosterone, erythromycin, cepoperazone, ceftriazone, itraconazole, ketoconozole, aureobasidin A, fluoroquinolone, lidocaine and bupivacaine.

Influx transport enhancement may be accomplished by the utilization of an agent selected from, though not limited to, short-chain sphingolipids, short-chain sphingomyelins and medium-chain glycerides.

Chemical penetration enhancement may be accomplished by the utilization of an agent selected from, but not limited to, a chitosan, benzalkonium chloride (BAK), sodium caprate, caprylic glycerides, capric glycerides, a phospholipids, lysophosphatidyleholine, didecanoylphosphatidylcholine, oleic acid, propylene glycol and PEG-8.

Physical penetration enhancement may be accomplished by the utilization of a technology selected from, though not limited to, iontophoresis, electroporation, phonoporatic energy, ultrasonic energy, high frequency waves, magnetic energy, electromagnetic energy, thermal energy and laser energy.

Biological penetration enhancement may be accomplished by the utilization of a class of agents selected from, but not limited to, modulators of intercellular tight junction expression, modulators of endocytosis-mediated delivery, modulators of receptor-mediated delivery and mediators of carrier-delivery. Biological penetration enhancement may be accomplished by the utilization of tissue permeability enhancers selected from the group consisting of hyaluronidase and hyaluronidase analogs.

Drug encapsulation technology may be accomplished by the utilization of a technology selected from, though not limited to, gels, hydrogels, micro/nanoparticles, micro/nanocapsules, dendrimers, micelles, emulsions, microemulsions, and liposomes.

Drug reservoir technology may be accomplished by the utilization of a technology selected from, though not limited to, inserts, drug soaked contact tenses, drug soaked collagen shields, particle-laden solid inserts, hollow core reservoirs, degradable polymer reservoirs, non-degradable polymer reservoir, combined degradable and non-degradable polymer reservoirs, ocular surface reservoirs, subconjunctival reservoirs and scleral reservoirs.

Drug convection or pumping technology is preferably accomplished by the utilization of a technology selected from, but not limited to, ocular surface pumps, ocular surface spraying systems, subconjunctival pumps and scleral pumps.

### EXAMPLES

Studies were done on rats as model-animals and enhanced intraocular drug penetration was achieved with different PlGF forms, different administered doses, different routes of administration, and at different times of intervention related to the time of model-drug administration. PlGF have provided significant enhancement effect over wide rang of doses. However, the preferred dose per each intervention was 5 ng per dose, diluted in buffered physiological solution from stock solution of 5 ng per microliter. The effectiveness was not limited to one isomer, but large scale experiments were done with the PlGF-1 isomer. Similarly, penetration enhancement after PlGF intervention was shown for various model-drugs, but large scale experiments were done with the 40 kDa fluorescent-labeled dextran solution with different concentrations. In all experiments, animals were sacrificed at different time intervals following the intervention and eyes were enucleated, sectioned following snap-freeze, and processed for evaluation by fluorescent microscopy to determine the extent of drug penetration through ocular barriers.

### Example 1

Intervention: PlGF was administered to the vitreous cavity and fluorescent labeled dextran solution was injected intravenously. In the control group animals, the same intervention was done but with the administration of buffered physiological solution (BPS) instead of PlGF.

Results: Special attention was made to obtain similar sections through the central retinal blood vessels for both of the PlGF treated and control animals. While the drug was seen to be confined to the lumen of blood vessels in the control group, the same retinal vessels have show significant leak of drug in the PlGF treated group, with subsequent drug penetration to the retina.

### Example 2

Intervention: PlGF was administered topically on the ocular surface followed by topical administration of the fluorescent-labeled dextran solution. In the control group animals, the same intervention was done but with the administration of BPS instead of PlGF.

Results: In the control group animals there was no drug penetration through the surface epithelium of the cornea and conjunctiva. After the PlGF intervention numerous sites of drug penetration between adjacent cells of the ocular surface epithelium were observed. Consequently, there was a remarkable drug penetration to the corneal stroma, deep penetration through the conjunctiva and penetration to the subconjunctival space. Furthermore, collecting vessels loaded with drug were leaking and releasing the drug at periocular, episcleral/scleral and choroidal sites, with drug penetration into the ciliary body and anterior retina.

### Example 3

Intervention: PlGF was administered to the anterior subconjunctival space followed by administration of the fluorescent-labeled dextran solution to the anterior subconjunctival space. In the control group animals, the same intervention was done but with the administration of BPS instead of PlGF.

Results: Compared to minimal intraocular drug penetration in the control group animals, there was a massive intraocular penetration in the group with PlGF intervention with remarkable drug accumulation in the ciliar body, anterior retina and corneal endothelial cells. Blood vessels loaded with the drug and leaking were seen again in the PlGF treatment group.

### Example 4

Intervention: PlGF was administered to the posterior subconjunctival space followed by administration of the fluorescent-labeled dextran solution to the posterior subconjunctival space. In the control group animals, the same intervention was done hut with the administration of BPS instead of PlGF.

Results: There was again a minimal intraocular drug penetration in the control group. On the contrary, numerous sites of leaky choroidal vessels were seen with massive release of the drug. Furthermore, drug was penetrating the RPE cell line in between adjacent cells and penetrating the anterior and posterior retina.

### Example 5

Intervention: PlGF was administered to the intravitreal space while the fluorescent-labeled dextran solution was administered to the subconjunctival space. In the control group animals, the same intervention was done but with the administration of BPS instead of PlGF.

Results: Massive opening of junctions between adjacent RPE cells was seen in the PlGF treated group with consequent drug penetration to deep layers of the anterior and posterior retina. On the contrary, intraocular drug penetration in the control group remained minimal.

Modifications and variations of the method and composition of the present invention for enhanced intraocular drug penetration will be obvious to those skilled in the art from the foregoing detailed description. Such modifications and variations are intended to come within the scope of the appended claims.

It will now be apparent to those skilled in the art that other embodiments, improvements, details, and uses can be made consistent with the letter and spirit of the foregoing disclosure and within the scope of this patent, which is limited only by the following claims, construed in accordance with the patent law, including the doctrine of equivalents.

1. A method for enhanced drug penetration to ocular and periocular tissues, comprising the administration of an ocular therapeutic agent, together with co-administration of a permeability-increasing factor of ocular surface and blood-ocular barriers.

2. The method of claim 1, wherein said permeability-increasing factor is a stimulator of receptor-1 of the vascular endothelial growth factor.

3. The method of claim 1, wherein said permeability-increasing factor is an effective amount of placental growth factor.

4. The method of claim 3, wherein said placental growth factor is PlGF-1.

5. The method of claim 3, wherein said placental growth factor is an effective PlGF isomer other than PlGF-1.

6. The method of claim 1, wherein said co-administration is a simultaneous administration.

7. The method of claims 1, wherein said co-administration is a non-simultaneous administration that ensures optimal effectiveness.

8. The method of claims 1, wherein the permeability-increasing factor is administered through a route that is selected from the group consisting of topical ocular, subconjunctival, periocular, intracameral and intravitreal routes of administration.

9. The method of claim 1, wherein the ocular therapeutic agent is administered through a route that is selected from the group consisting of: topical ocular, subconjunctival, periocular and non-ocular routes of administration.

10. The method of claim 1, additionally comprising at least one method for enhancement of drug penetration, said method selected from the group consisting of: efflux inhibition, influx transport enhancement, chemical penetration enhancement, physical penetration enhancement, biological penetration enhancement, drug encapsulation technology, drug reservoir technology and drug convection or pumping technology.

11. The method of claim 10, wherein said efflux inhibition is by the utilization of a p-glucoprotein inhibitor.

12. The method of claim 11, wherein said glucoprotein inhibitor is an agent selected from the group consisting of: verapamil, bepridil, dilatiazem, felodipine, nifedipine, nisoldipine, nitrendipine, tiapamil, vincristine, actinomycin D, colchicines, daunorubicin, etoposide, mitomycin C, mithramycin, podophyllotoxin, puromycin, taxol, topotecan, triamterene, vinblastine, cyclosporine A, cyclosporine H, tacrolimus, sirolimus, aldosterone, clomiphene, cortisol, deoxycorticosterone, dexamethasone, prednisone, progesterone analogs, tamoxifen, hydrocortisone, testosterone, erythromycin, cepoperazone, ceftriazone, itraconazole, ketoconozole, aureobasidin A, fluoroquinolone, lidocaine and bupivacaine.

13. The method of claim 10, wherein said influx transport enhancement is by the utilization of an agent selected from the group consisting of: short-chain sphingolipids, short-chain sphingomyelins and medium-chain glycerides.

14. The method of claim 10, wherein said chemical penetration enhancement is by the utilization of an agent selected from the group consisting of: a chitosan, benzalkonium chloride (BAK), sodium caprate, caprylic glycerides, capric glycerides, a phospholipids, lysophosphatidylcholine, didecanoylphosphatidylcholine, oleic acid, propylene glycol and PEG-8.

15. The method of claim 10, wherein said physical penetration enhancement is by the utilization of a technology selected from the group consisting of: iontophoresis, electroporation, phonoporatic energy, ultrasonic energy, high frequency waves, magnetic energy, electromagnetic energy, thermal energy and laser energy.

16. The method of claim 10, wherein said biological penetration enhancement is by the utilization of a class of agents selected from the group consisting of: modulators of intercellular tight junction expression, modulators of endocytosis-mediated delivery, modulators of receptor-mediated delivery and mediators of carrier-delivery.

17. The method of claim 10, wherein said biological penetration enhancement is by the utilization of tissue permeability enhancers selected from the group consisting of hyaluronidase and hyaluronidase analogs.

18. The method of claim 10, wherein said drug encapsulation technology is by the utilization of a technology selected from the group consisting of: gels, hydrogels, micro/nanoparticles, micro/nanocapsules, dendrimers, micelles, emulsions, microemulsions, and liposomes.

19. The method of claim 10, wherein said drug reservoir technology is by the utilization of a technology selected from the group consisting of inserts, drug soaked contact lenses, drug soaked collagen shields, particle-laden solid inserts, hollow core reservoirs, degradable polymer reservoirs, non-degradable polymer reservoir, combined degradable and non-degradable polymer reservoirs, ocular surface reservoirs, subconjunctival reservoirs and scleral reservoirs.

20. The method of claim 10, wherein said drug convection or pumping technology is by the utilization of a technology selected from the group consisting of ocular surface pumps, ocular surface spraying systems, subconjunctival pumps and scleral pumps.
